(19) United States
(12) Patent Application Publication
(10) Pub. No.: US 2018/0011026 A1
(43) Pub. Date: Jan. 11, 2018

(54) TRACE ELEMENTS DETECTION TEST PAPER AND PREPARATION METHOD THEREOF

(71) Applicant: Baby Care Development Technologies Corporation, City of Industry, CA (US)

(72) Inventor: Jie Xu, Harbin (CN)

(21) Appl. No.: 15/643,353

(22) Filed: Jul. 6, 2017

(30) Foreign Application Priority Data
Jul. 6, 2016 (CN) 201610528041.7

(51) Int. Cl. G01N 21/78; B05D 1/18; G01N 33/84; G01N 21/77

(52) U.S. Cl. CPC G01N 21/78; G01N 33/84; B05D 1/18; G01N 2021/7759

(57) ABSTRACT

The disclosure provides a trace element detection test, whose items are spliced by different variety of strips, including: routine urine test paper, test paper, calcium iron and zinc test, each block corresponding to each color recognition test items test results of paper. The trace element test paper of the invention can conveniently and comprehensively judge the urine routine items in children's urine and the content of trace elements such as calcium, iron and zinc whether normal or not. The preparation method of the calcium detection test paper is simple and effective, and has the advantages of low one-time use, convenient use and fast and accurate detection result.

### CROSS REFERENCE TO RELATED APPLICATIONS

This application claims the benefit and priority of Chinese Patent Application No. 201610528041.7, filed on Jul. 6, 2016, which is incorporated herein by reference in its entirety.

### TECHNICAL FIELD

The invention relates to a trace element test paper and a preparation method thereof.

### BACKGROUND

During the growth and development of children, trace elements are necessary, and the content of trace elements in children can be inferred by the content of trace elements in urine, and then the nutritional status of children can be analyzed.

The test of trace elements in children is comprehensive to the content of trace elements and convenient comprehensive judgment in children urine and urine calcium, iron and zinc projects are normal growth period, trace elements are necessary requirements, the contents of trace elements in the content of trace elements in urine of children body and push off, also can analysis of the nutritional status of children.

### SUMMARY

The technical problem to be solved by the disclosure is to provide a trace element test paper composite, which can conveniently and comprehensively judge whether the routine urine items in a person's urine and the content of trace elements such as calcium, iron and zinc are normal.

In order to solve the technical problem, the disclosure provides a trace element test paper composite including a variety of test papers for testing different substances. The trace elements test composite may include a urine test paper, a calcium detection test paper, an iron detection test paper, and a zinc detection test paper, wherein the urine test paper, the calcium detection test paper, the iron detection test paper, and the zinc detection test paper provide result indications according to different color indication schemes.

Preferably, each test paper in the trace elements test paper composite has a length of 5-30 mm for each test item, a width of 5-20 mm, and a thickness of 1-5 mm.

In some embodiments, the urine test paper detects at least a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

In some embodiments, the calcium detection test paper comprises porous nanomaterials having a porosity is 40%-80%, wherein calcium test materials are disposed on the porous nanomaterials.

In some embodiments, the calcium detection test paper comprises a component for detecting excessive calcium level and a component for detecting calcium deficiency, wherein the component for detecting excessive calcium level provides an indicator in a blue color when a normal calcium level is detected and provides an indicator in a red color when an excessive calcium level is detected, wherein the component for detecting calcium deficiency provides an indicator in a blue color when calcium deficiency is detected and provides an indicator in a red color in when a normal calcium level is detected.

The disclosure also provides a method for manufacturing a calcium detection test paper:

(1) Preparation of impregnating solution: 3.5 g-4.5 g buffer, 50 g-100 g surfactant, 2.5 g-5.5 g masking agent, 0.1 g-0.9 g calcium indicator and 0.01 g-1.5 g EDTA are added to volumetric flask, and the volume is fixed to 1000 mL, wherein, the buffer is prepared with sodium hydroxide, and the surface active agent is a combination of polyvinylpyrrolidone and polyethylene glycol, and the masking agent is a combination of sodium tartrate and 8-hydroxyquinoline;
(2) The porous nanometer material is soaked in the dipping liquid, soaked 20 min, taken out and dried in an oven, and the oven temperature is 30-50 degrees Celsius.

Preferably, a method for the preparation of a calcium detection test paper for the present invention; step (1) the pH of the impregnating solution is 8-13.

The disclosure also provides a method for manufacturing a zinc detection test paper, the manufacturing method is to soak a filter paper in a solution, drying and cutting the filter paper. The solution comprises V acid, sodium thiocyanate, and crystal violet, the acid may include hydrochloric acid and/or sulfuric acid.

Preferably, a method for manufacturing a zinc detection test paper according to various embodiments of the disclosure includes the following steps:

(a) Formulated solution: add a mixture of strong acid, sodium thiocyanate and crystalline violet to a volumetric flask, and hold it to 1000 mL;

(b) Immerse the filter paper completely in the solution, then soak it out and dry in the oven;

(c) The zinc ion in the dried urine is detected and the filter paper is cut into test paper strips.

Further, a method of generating a standard color card for the zinc detection test paper is provided. The method comprises recording different color indications from the zinc detection test paper based on additions of different content of zinc to the zinc detection test component.

The trace element test paper of the invention can conveniently and comprehensively judge the urine routine items in children's urine and the content of trace elements such as calcium, iron and zinc whether normal or not. The preparation method of the calcium detection test paper is simple and effective, and has the advantages of low one-time use, convenient use and fast and accurate detection result.

### BRIEF DESCRIPTION OF THE DRAWINGS

FIG. 1 is a schematic diagram of a test paper according to some embodiments of the disclosure.

Embodiments of the present disclosure and their advantages may be understood by referring to the detailed description herein. It should be appreciated that reference numerals may be used to illustrate various elements and features provided in the figures. The figures may illustrate various examples for purposes of illustration and explanation related to the embodiments of the present disclosure and not for purposes of any limitation.

### DETAILED DESCRIPTION

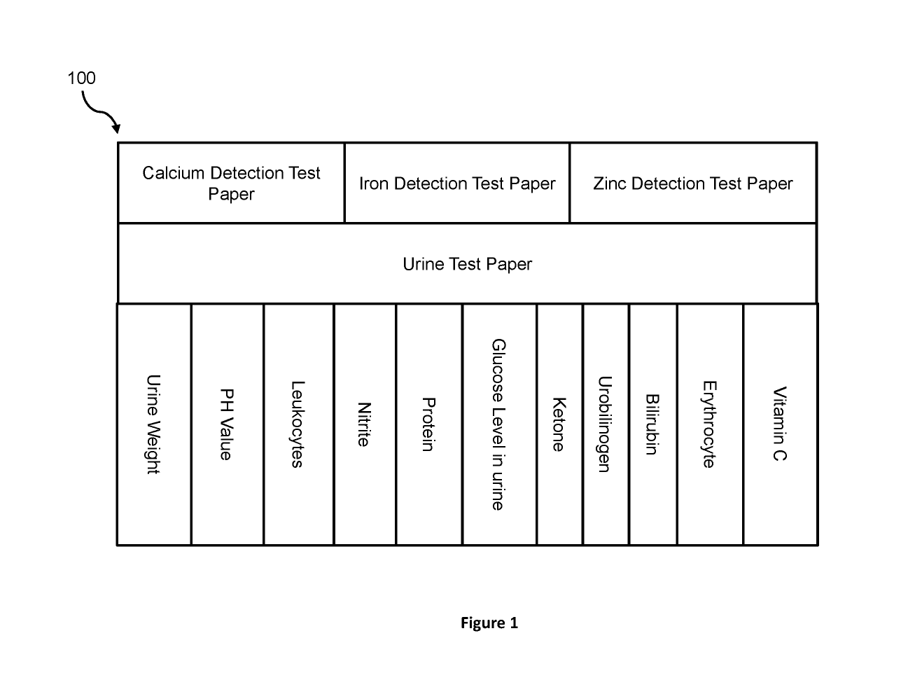

As shown in FIG. 1, a trace elements detection test paper composite 100 according to various embodiments of the disclosure comprises may test a variety of trace elements by using different test items test paper, For example, the trace elements test composite may include a urine test paper, a calcium detection test paper, an iron detection test paper, and a zinc detection test paper, wherein the urine test paper, the calcium detection test paper, the iron detection test paper, and the zinc detection test paper provide result indications according to different color indication schemes.

Preferably, each test paper in the trace elements test paper composite according to various embodiments of the disclosure has a length of 5-30 mm for each test item, a width of 5-20 mm and a thickness of 1-5 mm.

Further, the urine test paper detects at least a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

Further, the calcium detection test paper comprises porous nanomaterials having a porosity is 40%-80%, wherein calcium test materials are disposed on the porous nanomaterials.

Furthermore, the calcium detection test paper comprises a component for detecting excessive calcium level and a component for detecting calcium deficiency.

Further, the calcium excess test paper shows a color of blue when the content of calcium is normal, and shows a color of purple/red when the calcium level is excessive.

Further, the calcium deficiency test paper shows a color of blue when there is calcium deficiency, and shows a color of purple/red when the calcium content is normal.

The disclosure also provides a preparation method of a calcium element test paper:

(1) Preparation of impregnating solution: 3.5 g-4.5 g buffer, 50 g-100 g surfactant, 2.5 g-5.5 g masking agent, 0.1 g-0.9 g calcium indicator, and 0.9 g-1.5 g Ethylenediaminetetraacetic acid (EDTA) are added to volumetric flask, and the volume is fixed to 1000 mL, wherein, the buffer is prepared with sodium hydroxide, and the surface active agent is a combination of polyvinylpyrrolidone and polyethylene glycol, and the masking agent is a combination of sodium tartrate and 8-hydroxyquinoline;

(2) The porous nanometer material is soaked in the impregnating solution, soaked for 20 min, taken out and dried in an oven, and the oven temperature is 30-50 degrees Celsius.

Preferably, a method for the preparation of a calcium detection test paper for the present invention; step (1) the pH of the impregnating solution is 8-13.

### Embodiment 1

The disclosure provides a method for manufacturing a calcium deficiency detecting test paper for urine, comprising the following steps:

(1) Preparation of impregnating solution: 3 grams (g) of sodium hydroxide, 25 g of polyvinylpyrrolidone, 30 g of polyethylene glycol, 5.5 g of tartaric acid, 0.05 g of calcium indicator, and 0.9 g of Ethylenediaminetetraacetic acid (EDTA) into the volumetric flask, and use distilled water to fix the volume to 1000 mL;

(2) Making porous nano material: the specific surface area is 1200 m2/g, soaking 1 g of activated carbon having an average pore diameter of 2.5 nm in concentrated nitric acid of 70% concentration, stirring at 65 degrees Celsius for 2 hours, filtering, washing and drying. Put 1 g of acrylamide into 50 ml of distilled water, and heat at 45 degrees Celsius until completely dissolved. Then, in order, add 50 ml of methanol, a small amount of ammonium persulfate, stir evenly, pour into the container containing the activated carbon, stir for 10 h at low temperature. Then, the container was heated in a nitrogen atmosphere at a constant temperature of 70 degrees Celsius for 4 h, and finally washed with ethanol for several times before drying. Compress and mold the generated porous nano polyacrylamide activated carbon composite material using a mold. Finally, the test strip was made using the compressed and molded porous nano polyacrylamide activated carbon composite material having a length of 15 mm, a width of 5 mm, and a thickness of 2 mm;

(3) The porous nano material is soaked in the impregnating solution, soaked for 5 min, removed and dried in an oven, and the oven temperature is 60 degrees Celsius.

The test strip is immersed in test liquid for 1 second and then taken out, observe the color of the detection end of the test strip after 10 seconds, the color blue represents a deficiency of Calcium, the purple red color represents the calcium level is normal.

### Embodiment Two

The present embodiment provides a method for preparing a test paper for detecting excessive calcium in children's urine, comprising the following steps:

(1) Preparation of impregnating solution: 3 g of sodium hydroxide, 25 g of polyvinylpyrrolidone, 30 g of polyethylene glycol, 3 g of tartaric acid, 1.5 g of calcium indicator, and 1.5 g of EDTA into the volumetric flask, and distilled water to 1000 ml;

(2) Making porous nano material: the specific surface area is 1200 m2/g, soak 1 g of activated carbon having an average pore diameter of 5 nm in concentrated nitric acid of 70% concentration, stirring at 65 degrees Celsius for 2 hours, filtering, washing and drying. Add 1 g acrylamide into 50 ml of distilled water, and heat at 45 degrees Celsius until completely dissolved. Then, in order, add 50 ml of methanol, a small amount of ammonium persulfate, stir evenly, pour into the container containing activated carbon, stir for 10 h at low temperature. Then, the container was heated in a nitrogen atmosphere at a constant temperature of 70 degrees Celsius for 4 h, and finally washed with ethanol for several times before drying. Made of porous nano polyacrylamide activated carbon composite material, the material was compressed and molded by mould. Finally, the test strip was made with length 15 mm, width 5 mm and thickness 2 mm;

(3) The porous nano material is dipped in the impregnating solution, soaked 5 min, taken out and dried in an oven, and the oven temperature is 60 degrees Celsius;

The test strip is immersed in test liquid, after 1 seconds out, observe the color of the testing end after 10 seconds, when the color of the strip is blue, the calcium content is normal, and when the color of the strip is purple, the calcium content is excessive.

The disclosure also provides a preparation method of zinc detection test, the manufacturing method is to soak a filter paper in a solution, drying and cutting system, the solution comprising strong acid, sodium thiocyanate, and crystal violet, the strong acid may include hydrochloric acid or sulfuric acid.

### Embodiment Three

The preparation method of the zinc detection test paper:

(1) Preparing a solution: adding 10-40 g of sulfuric acid, 5-30 g of sodium thiocyanate, and 0.1-2 g of crystal violet to volumetric flask, constant volume to 1000 ml;

(2) Immerse the filter paper completely in the solution, then take it out and dry in the oven;

(3) After drying, the filter paper can now detect zinc ions in urine, and the filter paper is cut into test paper strips.

### Embodiment Four

The preparation method of the zinc element test paper:

(1) Preparation of solution: adding 5-30 g of hydrochloric acid, 5-30 g of sodium thiocyanate, and 0.1-2 g of crystal violet to volumetric flask, and constant volume to 1000 ml;

(2) Immerse the filter paper completely in the solution, soak for 10 min, then remove and dry in the oven;

(3) After drying, the filter paper can now detect zinc ions in urine, and the filter paper is cut into test paper strips.

### Embodiment Five

According to the color changes of the zinc detection test paper by adding liquids with different known zinc content to the zinc detection test paper, a new element detection standard color card is made. Include the following steps:

(1) Prepare different liquids having the following zinc concentration 0 g/L, 0.1 g/L, 0.15 g/L, 0.2 g/L, 0.5 g/L, 1 g/L, 1.1 g/L, 1.2 g/L, 1.3 g/L;

(2) Test papers prepared by embodiment 1 or embodiment 2 are dipped in the prepared liquids, then removed and dried for 2 seconds, and the colors are detected after 20 seconds

(3) According to the test papers' colors, prepare the color card, color card color is green, blue, blue and purple three order.

The trace element test paper of the disclosure can conveniently and comprehensively judge the urine routine items in children's urine and the content of trace elements such as calcium, iron and zinc whether normal or not. The preparation method of the calcium detection test paper is simple and effective, and has the advantages of low one-time use, convenient use and fast and accurate detection result.

The present disclosure, the accompanying figures, and the claims are not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure.

1. A trace elements detection test paper composite, comprising:
a urine test paper;
a calcium detection test paper;
an iron detection test paper; and
a zinc detection test paper,
wherein the urine test paper, the calcium detection test paper, the iron detection test paper, and the zinc detection test paper provide result indications according to different color indication schemes.

2. The trace elements detection test paper composite of claim 1, wherein each of the urine test paper, the calcium detection test paper, the iron detection test paper, and the zinc detection test paper has a length of between 5 mm and 30 mm, a width of between 5 mm and 20 mm, and a thickness of between 1 mm and 5 mm.

3. The trace elements detection test paper composite of claim 2, wherein the urine test paper detects at least a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

4. The trace elements detection test paper composite of claim 2, wherein the calcium detection test paper comprises porous nanomaterials having a porosity between 40% and 80%, wherein calcium test materials are disposed on the porous nanomaterials.

5. The trace elements detection test paper composite of claim 4, wherein the calcium detection test paper comprises a component for detecting excessive calcium level and a component for detecting calcium deficiency, wherein the component for detecting excessive calcium level provides an indicator in a blue color when a normal calcium level is detected and provides an indicator in a red color when an excessive calcium level is detected, wherein the component for detecting calcium deficiency provides an indicator in a blue color when calcium deficiency is detected and provides an indicator in a red color in when a normal calcium level is detected.

6. A method for manufacturing a calcium detection test paper, the method comprising:
preparing an impregnating solution by adding 3.5 g-4.5 g of buffer, 50 g-100 g of surfactant, 2.5 g-5.5 g of masking agent, 0.01 g-1.5 g of calcium indicator, and 0.9 g-1.5 g of Ethylenediaminetetraacetic acid to volumetric flask, wherein the solution has a volume of approximately 1,000 mL, wherein, the buffer comprises sodium hydroxide, and the surfactant comprises a combination of polyvinylpyrrolidone and polyethylene glycol, and the masking agent comprises a combination of sodium tartrate and 8-hydroxyquinoline;
soaking porous nanomaterials in the impregnating solution for approximately 20 minutes; and
drying the porous nanomaterials in a temperature between 30 degrees Celsius and 50 degrees Celsius.

7. The method of claim 6, wherein the impregnating solution has a pH value of between 8 and 13.

8. A method of manufacturing a zinc detection test paper, the method comprising:
soaking a filter paper in a solution; and
drying the soaked filter paper,
wherein solution comprises acid, sodium thiocyanate, and crystal violet, wherein the acid comprises hydrochloric acid or sulfuric acid.

9. The method of claim 8, further comprising:
generating the solution by adding a mixture of the acid, the sodium thiocyanate, and the crystalline violet to a volumetric flask, wherein the mixture has a volume of approximately 1000 mL;
disposing the filter paper entirely into the solution;
drying the filter paper; and
trimming the filter paper into strips of zinc detection test papers.

10. The method of claim 8, further comprising generating a color standard card for the zinc detection test paper by recording different color indications from the zinc detection test paper based on additions of different content of zinc to the zinc detection test component.
